(19) United States
(12) Patent Application Publication
(10) Pub. No.: US 2006/0183205 A1
(43) Pub. Date: Aug. 17, 2006

(54) Method for controlling molecular weight and distribution of biopolymers

(76) Inventors: Laurent Masaro, Montreal (CA); Patrick Lapointe, Montreal (CA); Jean-Charles Jacques-Gayet, Montreal, QC (US)

Correspondence Address: OGILVY RENAULT LLP, 1981 MCGILL COLLEGE AVENUE, SUITE 1600, MONTREAL, QC, H3A2Y3, CA

(21) Appl. No.: 10/544,025

(22) PCT Filed: Jan. 29, 2004
(86) PCT No.: PCT/CA04/00132

§ 371 Date: Feb. 14, 2006

(30) Foreign Application Priority Data
Jan. 29, 2003 (US) 60443162

(51) Int. Cl. C12P 7/62; C08J 3/28

(52) U.S. Cl. 435/135; 522/1

(57) ABSTRACT

A method has been developed for the control of both molecular weight and molecular weight distribution of polyhydroxyalkanoates (PHAs) produced by bacterial strains. The control is exerted once the biopolymer has been accumulated intracellularly through a fermentation process by a chemical, enzymatic or irradiation treatment. More particularly, the control of both molecular weight and molecular weight dispersity is achieved by keeping the biopolymer in its native state during the whole process. This implies that no or little or no crystallization has occurred. In another embodiment, the decrease of the molecular weight can be achieved during the extraction and purification process.

### TECHNICAL FIELD

The present invention relates to controlling both molecular weight and molecular weight distribution of polyhydroxyalkanoates produced by fermentation.

### BACKGROUND ART

Polyhydroxyalkanoates (PHAs) are natural polyesters produced by microorganisms, bacteria and algea, as intracellular energy storage material in the presence of excess of carbon source under unfavorable growth conditions. Since PHAs are natural and entirely biodegradable when placed in compost conditions, they have attracted much interest in the last decades. In addition, these biopolymers can be easily processed with conventional equipments to produce thermoplastics in order to replace non-environmental friendly polymers and resins to reduce solid wastes. Some PHAs already developed are polyhydroxybutyrate (PHB) and poly(hydroxybutyrate-co-hydroxyvalerate) (PHBV), still currently the most common used PHAs. However, today new developments have allowed the production of these biopolymers at much more competitive prices that enhance the attraction to this class of biopolymers.

Different ways have been developed to produce PHAs. The most common and widespread is by bacterial fermentation. In fact, over 90 gram-negative and gram-positive bacteria were reported to accumulate PHA type biopolymers (Steinbuchel, Biomaterials, Stockton Press, New-York, 125-213, 1991). Microorganisms produce PHAs using a R-β-hydroxyacyl-CoA as direct metabolite substrate for the PHA synthase. Therefore, the resulting biopolymer is 100% isotactic, poly[R-(3)-hydroxyalkanoates] having chain lengths ranging from C₃ to C₂₀ depending on the nutrients and the strategy used to feed them.

Another characteristic of these biopolymers produced by microorganisms is their high molecular weight, generally larger than 500,000 g/mol. Azotobacters accumulate PHB of 8×10⁵ to 2×10⁶ g/mol, A. eutrophus accumulates PHB of 6×10⁴ to more than 1×10⁶ g/mol. Once the growth mechanism of the microorganism is well characterized, understood and controlled, it is common to obtain biopolymers with molecular weight in the order of or above 1,000,000 g/mol.

Transgenic plants can produce PHAs with narrow distribution. This way of production is far less developed than fermentation because of longer research efforts that are still needed. The physico-chemical characteristics of the 4 biopolymer thus produced seem to be dependent on the plant it grows in. For example, genetically modified canola plant can provide PHA with molecular weight of 686,348 g/mol and an index of poly-dispersity (a measure of the breadth of a molecular weight distribution) equal to 2.47, whereas soybean plant provides PHA of molecular weight 209,685 g/mol and an index of poly-dispersity of 2.10.

Another way to produce PHAs is by chemical synthesis. Ring opening of β-butyrolactones is the most carried out polymerization synthesis process. Important distinctions have to be stated between this method of synthesis and the previous ones stated above. First, biopolymers are no longer systematically isotactic poly[R-(3)-hydroxyalkanoates]. Depending on the catalyst employed and the stereochemical configuration of the starting material, the biopolymer can be either isotactic, syndiotactic or atactic. Second, the tacticity seems to affect the molecular weight. Isotactic PHAs were obtained with molecular weights lower than 500,000 g/mol, whereas atactic PHAs were obtained with molecular weights above 500,000 g/mol.

Transformation of a polymer into a plastic is a process achieved by melting and mixing the polymer through an extruder, therefore the compound has to be sufficiently viscous to maintain its shape, i.e., not to flow. As the viscosity is proportional to the polymer's molecular weight, high molecular weight polymers are required for thermoplastic applications.

Pharmaceutical applications also require high molecular weight polymers. For example, oral tablets are made with high molecular weight polymers, such as cellulose and its derivatives, because of their high compaction properties. Resorption of the polymer matrix is not a concern in this particular situation as the polymer is naturally excreted if not degraded in-vivo. However, the resorption became an important issue for other pharmaceutical and biomedical applications such as implants, sutures, prosthesis, etc. In the case of biodegradable polymers, it is well known that the resorption capacity is inversely proportional to the molecular weight. Low molecular weight polymers are biodegraded rapidly while high molecular weight polymers need more time to be resorbed. Molecular weight dispersity affects also the resorption because a large poly-dispersity implies a broader distribution curve. Therefore, higher molecular weight polymers that resorb slowly are present. Consequently, the control of the molecular weight and molecular weight poly-dispersity is a major concern in order to manage the degradation of the biopolymers, when introduced, for example, in a living body.

As mentioned above, PHA granules are intracellular occlusions. Therefore, the extraction and purification processes are difficult and critical steps in the obtainment of the biopolymer from what is commonly called non-PHA cell materials (NPCM). Several methods have been tested and described during the last years. The main objectives of these studies were to recover the biopolymer from the NPCM without altering it and without increasing the production costs. However, certain protocols have reported severe degradation of the biopolymer. Prolonged exposure to halogenated solvents and heat induces degradation.

It is known that extraction with potent chemical agent can reduce the molecular weight of the biopolymer in addition to digest the NPCM. Prolonged treatment in the presence of strong acid, base, enzyme or eventually oxidizing and reducing agents are known to degrade polyesters.

Few ways to monitor molecular weight and molecular weight poly-dispersity do exist. However, they are very complex to implement. Snell et al. described a genetically engineered organism to produce PHAs with determined molecular weight in International Patent Pub No. WO 98/04713.

International Patent No. WO 97/07153 reports that addition of poly (ethylene glycol) (PEG) to the culture media allows control of PHAs' molecular weight of PHAs. In such case, PEG had to be incorporated in the culture medium before the accumulation of biopolymers in cells, otherwise a double distribution will result. Unfortunately, this constraint has an impact on the concentration of viable cells.

Based on the above-described state of the art, there is still a large place for improvement in producing PHAs with a simple and efficient method to control both molecular weight and molecular weight distribution of PHAs produced by fermentation process.

### DISCLOSURE OF INVENTION

One object of the present invention is to provide a method for preparing biopolymers with controlled molecular weight or molecular weight distribution comprising at least one chemical, enzymatic or irradiation treatment for selected combination of time and temperature to allow formation of a biopolymer having targeted molecular weight or weight distribution.

The biopolymer, which can be in an amorphous state or have a low degree of crystallization, can also be in an aqueous latex suspension.

The biopolymer may be selected from the group of polyhydroxyalkanoate(PHA), polylactic acid(PLA), poly(lactic-co-glycolic) acid(PLGA), polyglycolic acid(PGA), polycaprolactone(PCL), adipic acid, aminocaproic acid, poly (butylene succinate), or a derivative or a mixture thereof.

Another object of the present invention is to provide a method for preparing biopolymers with controlled molecular weight or molecular weight distribution comprised of at least one chemical, enzymatic or irradiation treatment for a period of time between one minute and one hundred hours, at a temperature is between about 1 to 99° C., under conditions to obtain a biopolymer having a dispersity index which can be between about 1.5 and 2.5.

When a chemical treatment is used, it can be performed under acid conditions at pH between 0 and 7, or under basic conditions at pH between about 7 and 14.

When an enzymatic treatment is used, it can be performed with an enzyme depolymerase, and when an irradiation treatment is used, it can be performed by x-ray, microwave, or gamma irradiation.

When a chemical treatment is used, it may be performed with an oxidizing or a reducing agent for decreasing the molecular weight of the biopolymer.

For the purpose of the present invention the following terms are defined below.

The term “biopolymer” as used herein is intended to mean polymers obtained from natural and renewable sources and which mode of synthesis occurs naturally such as with plants or microorganisms.

The term “polymers” as used herein is intended to mean macromolecules synthesized by chemical reaction or obtained from petroleum sources, even if one of the components (monomer, precursor, etc.) is obtained from natural and renewable sources.

The terms “granules” and “particles” as used herein are intended to mean spheroids shaped biopolymer segments with particle size distribution between 0.1 and 10 μm, preferably between 0.2 and 5 μm.

The term “latex” as used herein is intended to mean a suspension of PHA granules and/or particles in an aqueous medium. The PHA granules can be either in their native state or re-suspended in water. The native PHA is defined as a granule of PHA, produced by bacterial fermentation, which was never precipitated, therefore its crystallization degree remains close to or slightly higher than it was in the bacteria, i.e., very weak. The latex may have the aspect of milk in color and texture, while the viscosity may be similar to that of water.

The term “chemical treatment” as used herein is intended to mean the action of acids, alkalies, surfactants, oxidizing or reducing agents or solvents likely to chemically react with PHAs by reducing their molecular weight and altering their molecular weight distribution.

The term “enzymatic treatment” as used herein is intended to mean the action of enzyme likely to chemically react with PHAs by reducing their molecular weight and thus changing their molecular weight distribution.

### MODES OF CARRYING OUT THE INVENTION

In accordance with the present invention, there is provided a method of producing polyhydroxyalkanoates (PHAs) with both controlled molecular weight and molecular weight distribution by chemical, enzymatic or irradiation treatment.

The Applicant has discovered that, for example, by subjecting a native latex solution of PHA to a chemical and/or enzymatic treatment, or irradiation treatment, both the molecular weight and molecular weight poly-dispersity of the biopolymer can be monitored and tailored.

In one embodiment of the present invention, at least one of a chemical or enzymatic treatment can be used to control both the molecular weight and molecular weight dispersity of a biopolymer before, during or after its synthesis or purification. The method may involve a chemical, enzymatic or irradiation treatment before, during or after the extraction and purification steps of the biopolymer.

In one other embodiment of the present invention, an irradiation treatment can be used to monitor both the molecular weight and molecular weight dispersity.

The biopolymer is preferably in its native state before, the chemical and/or enzymatic, or irradiation treatment, so as not to affect the molecular weight poly-dispersity of the resulting lower molecular weight PHAs.

According to another embodiment of the present invention, a biopolymer latex solution is prepared without using techniques or steps that will cause drying of the biopolymer. The biopolymer can be selected from, but is not limited to, the group of a polyhydroxyalkanoate(PHA), a polylactic acid(PLA), a poly(lactic-co-glycolic) acid(PLGA), a poly-glycolic acid(PGA), polycaprolactone(PCL), an adipic acid, an aminocaproic acid, a poly (butylene succinate), or a derivative or a mixture thereof.

In another embodiment of the present invention, the biopolymer latex solution is preferably prepared using separation techniques that avoid agglomeration of the biopolymer.

Also, the preparation of the biopolymer latex solution can be achieved using decantation, centrifugation or filtration that respect the previous embodiments, i.e., leads to a soup like solution and not to a solid like material. Surfactants can be used to prevent the agglomeration of PHA particles.

In one embodiment of the present invention, the preparation of a native biopolymer latex solution formed through a fermentation process can be achieved in a minimum period of time to avoid agglomeration of the biopolymer particles.

In still another embodiment of the present invention, the native biopolymer granules can be extracted from the bacterial cells at a temperature between 10 and 70° C. so as not to alter the interactions between the biopolymer granules and the medium.

In another embodiment of the present invention, the chemical and/or enzymatic treatment can be used to further purify the biopolymer from the NPCM residues by digesting them. Alternatively, the irradiation treatment can be achieved before lysing cells walls of the biopolymer producing bacteria.

The invention is applicable to control and decrease molecular weight without extensively increasing molecular weight distribution of any type of biopolymer, and particularly PHAs and derivatives thereof, produced by plants or microbial organisms either naturally or through genetic engineering, as well as chemically synthesized polymers.

According to one other embodiment of the present invention, the PHA biopolymers that may be used are polyesters composed of monomer units having the formula: 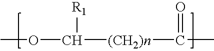
wherein n is an integer from 1 up to and including 5; R₁ is preferably H, an alkyl or alkenyl group. The alkyl and alkenyl side chains are preferably from C, up to C₂₀. A PHA biopolymers can be homopolymers, with the same recurring monomer unit, and/or copolymers with at least two different recurring monomer units. Statistically structured, random, block, alternating or graft copolymers may be used. The molecular weights of the PHA biopolymers are preferably in the range of 1,000 to 10,000,000 g/mol, preferably between 50,000 and 5,000,000 g/mol, and more preferably between 500,000 and 2,000,000 g/mol.

The orientation of the monomers among themselves can be head to head, head to tail or tail to tail.

The PHAs that can be used according to this invention include poly(3-hydroxybutyrate), poly(3-hydroxyvalerate), poly(3-hydroxyoctanoate), poly(4-hydroxybutyrate), medium chain length polyhydroxyalkaonates, poly(3-hydroxybutyrate-co-3-hydroxyvalerate), poly(3-hydroxybutyrate-co-4-hydroxybutyrate) and poly(3-hydroxybutyrate-co-3-hydroxyoctanoate). The copolymers of PHA, listed here above, may contain between 40 to 100% and preferably between 60 to 95% of the 3-hydroxybutyrate monomer.

According to this invention, the PHA concentration in the latex solution is from 0.01 up to 50%, preferably from 5 up to 40% and more preferably from 10 up to 30%. Concentrations are expressed in weight/volume. The latex can be obtained from a native biopolymer but cannot be resuspended from a dry powder. In the latter case, the high crystallinity of the biopolymer will deeply affect the molecular weight poly-dispersity of the resulting low molecular weight biopolymer. The origin of the biopolymer is also extended to include those returned to amorphous state.

According to the invention in its first aspect, the addition of a chemical and/or enzymatic agent, or the irradiation treatment of a native PHA latex solution is characterized by a decrease of the molecular weight of the biopolymer without any drastic increase in the molecular weight poly-dispersity.

Both phenomenon may be attributed to the fact that biopolymer chains scissions are purely static. In the case of a previously dried biopolymer, i.e., wherein the chemical and/or enzymatic chains are crystallized, scissions are first directed to amorphous regions. This phenomenon leads to an important increase in the molecular weight distribution.

The chemical reagent may be an acid or an alkali. Strong or weak acids and bases may be used. Further, they can be used with or without the addition of a buffer and/or a catalyst. The chemical reagent may also be an oxidizing or reducing agent.

The enzymatic reagent may include biopolymer depolymerases, such as polyhydroxyalkanoate depolymerase, obtained from natural or synthetic sources.

According to the present invention, the concentration of the acid or base, added to the latex solution, is between 0.01 N up to 10 N, preferably between 0.05 N up to 5 N and more preferably between 0.1 N and 2 N. In the case of an acid treatment, an acid solution containing one or several acid compounds—at least 2 up to several (10 or more)—at the same or different concentrations. The nature of the activity of the acid added can also vary. For example, an acid with a strong activity will be more efficient, resulting in shorter treatment time. The same can be applied to alkalies.

According to the present invention, the temperature for the chemical and/or enzymatic treatment may be between 5° C. up to 90° C., preferably between 10° C. up to 80° C. and more preferably between 20° C. and 70° C.

According to the present invention, the duration of the chemical and/or enzymatic treatment may be between a few minutes up to several hours. In fact, this parameter is strongly dependent on the ones mentioned above, i.e. the initial molecular weight of the native PHA and the desired final molecular weight.

According to the invention, the molecular weights of the biopolymers can be tailored for industrial, food, cosmetic and pharmaceutical applications for humans as well as animals.

The present invention will be more readily understood by referring to the following examples that are given to illustrate the invention rather than to limit its scope.

### EXAMPLE I

### Production of PHAs Latex Solution

Materials and Methods

Microorganism and Culture Media

The strain used for the production of PHA is Azotobacter salinestris (ATCC 49674). Azotobacter salinestris is a gram-negative bacterium related to Azotobacter chroococcum and is cultured in a medium as described above.

The fermentation inoculum consists of a pre-grown (18-24 h) culture with a corresponding cell dry weight of 1-5 g/l. Samples of quickly halted log growth phase are mixed with an equal volume of glycerol 30% (v/v) and stored in vials (1-2 ml) at −80° C. to constitute a working cells bank.

Potato Starch Hydrolysis

Potato tubers or peels are first washed and shredded. Water is then added to form 500-2000 g/l potato slurry depending on final glucose concentration desired. The resulting mixture may then be subjected to starch hydrolysis, which is a two step process. In the first one, called liquefaction, the starch slurry is heat treated (65-95° C. at 350 rpm for 30min-1 h), before being hydrolyzed to a maltodextrine solution with a heat-stable a-amylase enzyme preparation (Termamyl® 120L, Novo Nordisk) in presence of calcium ions.

This step is carried out directly in a steamed tank reactor vessel equipped with temperature, stirrer speed and pH adjustments, all of which being set at the following operating parameters: 90-100° C.; 200-350 rpm; pH=6.0-6.5 for a period of up to 60-120 min. The pH may be adjusted with calcium hydroxide to provide the necessary calcium ions. The second step, called saccharification, allows for further hydrolysis of the dextrines into glucose. It is performed with a 1,4-alpha-D-glucan glucohydrolase (AMG 300, Novo Nordisk) after setting the operating parameters as 55-60° C.; 200-250rpm; pH=4.2-4.8 for a period of 24-60 h. The degree of enzymatic hydrolysis may be determined with the use of a rapid analysis system for the glucose concentration (Biolyzer by Kodak, New Haven, Conn.).

Fed-Batch Culture

Fermentation is performed in a conventional controlled stirred tank reactor (STR) at 25-30° C. and pH=7.0. The fermentation media is the same as the one described above for the cultivation of the microorganism. The ferment is seeded with a 2-10% (v/v) fresh inoculum in active growth phase. The agitation and airflow rate are varied during the course of fermentation to maintain the dissolved oxygen level (DO) above 3-5% saturation and preferably around 5-10% saturation. Following a log phase of 4-10 h, it is necessary to maintain the glucose level by feeding with a hydrolyzed starch stock solution at a concentration of 20-80% w/v glucose at a variable feed rate in the range of 5-10 ml/l/h. Fish peptone, modified meat peptone, or yeast extract may be also supplied to the growth medium to enhance PHB synthesis. Peptones are thought to act as a PHA yield promotion factor at a concentration of between 0.05 to 0.2% (w/v). For best results, the peptone solution should be added at a rate proportional to the glucose supplement. It is also required to maintain a continuous supply of broth nutrient by feeding a concentrate of the fermentation medium throughout the growth phase. A typical feedstock may consist of a 4-20 times the initial broth concentration and should be supplied at a rate proportional to glucose feed solution. At the end of fermentation, cells are separated from the spent medium by centrifugation or filtration.

Polymer Extraction Method

PHA isolation consists in a step procedure, in which cells are sequentially separated, washed and then submitted to polymer extraction as described. Cells are washed once or twice in distilled water and membranes are broken by using hot mixture of NaOH and NH₄OH or NaOH, NH₄OH and SS or NaOH, NH₄OH and Triton™, or mechanically by glass beads or other shear forces or by heat treatment. PHA is then isolated using different approaches such as solvent extraction using chloroform or methylene dichloride or by digesting NPCM (non polymer cell material) using enzyme cocktail of protease, lipase and nuclease. PHA is finally recovered by centrifugation, differential centrifugation or filtration, and dried avoiding direct light exposure. Physical determination such as average molecular weight and poly-dispersity index may be carried out using standard procedures known in the art.

### EXAMPLE II

### Growth of A. salinestris and Production of PHA Following a Fed Batch Fermentation Strategy

An inoculum of A. salinestris (strain ATCC 49674) was grown aerobically in a 2 liters Fernbach™ flask containing 500 ml of previously described culture medium. The flask was incubated at 30° C. for 24 h with rotating agitation set at 250 rpm.

The resulting inoculum was then added to a 14 liters bioreactor (CHEMAP) containing 8 liters of the previously described fermentation medium. The fermentation was carried out at 30° C. in a fed-batch mode in the following conditions: 1) the pH was maintained at 7 using a concentrated solution of sodium hydroxide or sulfuric acid; 2) the aeration rate and the agitation speed were adjusted manually during the fermentation to maintain the level of oxygen above 5% and below 30% saturation. The maximum agitation speed reached was 610 rpm; 3) foam formation was controlled with the addition of MAZU™ (PPG Industries); 4) glucose was fed throughout growth phase from 20-80% w/v stock solution as obtained by starch hydrolysis, at a rate of approximately 5-10 ml/l/h; 5) spent nutrients were provided throughout growth phase by feeding a 4-20 times concentrated fermentation medium. Feed rate was approximately 5-10 ml/l/h. The fermentation was stopped after 30 hours.

The PHA was recovered using a modified method of Berger and colleagues (Biotechnology Techniques, 1989, 3:227-232). Cells were centrifuged 15 minutes at 3000×g and then washed twice in distilled water. 50 ml of methanol were added to an equivalent of 5 g (dry weight) of cells and vigorously mixed. The mixture was incubated 48 h at 40° C. and the cells were harvested by centrifugation at 3000×g for 15 minutes. The supernatant was discarded and 100 ml of chloroform was added to the pellet. The mixture was gently agitated and incubated at 40° C. for 24 h. 100 ml of distilled water was added to the chloroform mixture, carefully agitated and centrifuged at 3000×g for 15 minutes. The lower phase was recuperated and the soluble polymer precipitated with the addition of cold ethanol 95% under continuous agitation. The precipitated PHA obtained was recovered by filtration and dried at room temperature avoiding light exposure.

At the end of the fermentation, the cell biomass concentration was 30-40 g/l (dry weight), containing approximately 15-20 g/l of PHB/HV (92% HB and 8% HV) with a molecular weight of 1 million and a poly-dispersity index of 1.2.

### EXAMPLE III

### Production of copolymer PHB/HV Following a Co-Substrate Fedbatch Fermentation Strategy

A inoculum of A. salinestris (ATCC 49674) was grown aerobically in a 2 liters flask containing 500 ml of previously described culture medium supplemented with 30 mM sodium valerate. The culture was incubated at 30° C. for 24-30 h rotating agitation set at 250 rpm.

The fermentation parameters were similar to that described in Example I for the aeration rate, pH and dissolved oxygen level. Sodium valerate as well as glucose were added during the fermentation from a concentrate of 500 mM sodium valerate and 50% glucose in order to obtain a random copolymer of 3HB-3HV or a copolymer block. Depending on the feed strategy, copolymers were composed of 65 to 90% of HB and 10 to 35% of HV, with a MW of 1 million and P.I. of 1.2.

### EXAMPLE IV

### Reduction of the Molecular Weight of a Latex Solution Using NaOH

The concentration of the PHA in the latex obtained after fermentation and extraction, as described in Example I, is 15% weight/volume. The molecular weight of this biopolymer is 1,100,000 g/mol and its poly-dispersity equals 1.7, as characterized by size exclusion chromatography (SEC) with chloroform as eluant.

Five liters of the last solution is placed in a reactor, 110 ml of NaOH 10 N are added smoothly in order to obtain a solution 0.2 N, i.e., pH=13.3. The temperature is set up to 55° C., agitation between 300 and 400 rpm. After 5 hours, the solution is centrifuged, using a centrifuge that does not provide solid material but rather a soup like solution, i.e., without cake formation. The volume of this solution is completed to 5 L and centrifuged again. This operation is repeated another time in order to remove all trace of sodium hydroxyl and salts. The last centrifugation is achieved with a centrifuge that provides solid material, the later is placed in an oven overnight at 70° C. to obtain a pure and dry PHA with reduced molecular weight and molecular weight distribution.

The characteristics of this biopolymer determined by SEC are the following: molecular weight 500,000 g/mol; poly-dispersity 2.1.

### EXAMPLE V

### Reduction of Molecular Weight of Precipitated Biopolymer Using NaOH

The same experiments described in Example IV were repeated with a biopolymer that was dried during the purification and extraction steps. Instead of using a centrifuge that provides a soup like solution, a centrifuge that provides a solid material was used.

The characteristics of this biopolymer determined by SEC are the following: molecular weight 600,000 g/mol; poly-dispersity 8.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method for controlling at least one of molecular weight or molecular weight distribution of biopolymers comprising submitting said biopolymers to at least one of a chemical, an enzymatic or an irradiation treatment at selected combination of time and temperature to allow formation of a biopolymer having targeted molecular weight or weight distribution.

2. The method of claim 1, wherein said biopolymer is in an aqueous latex suspension.

3. The method of claim 1, wherein said biopolymer is in an amorphous state or has a weak degree of crystallization.

4. The method of claim 1, wherein said biopolymer is selected from the group of polyhydroxyalkanoate (PHA), polylactic acid (PLA), poly (lactic-co-glycolic) acid (PLGA), polyglycolic acid (PGA), polycaprolactone (PCL), adipic acid, aminocaproic acid, and poly (butylene succinate), or a derivative or a mixture thereof.

5. The method of claim 1, wherein said period of time is between 1 minute and hundred of hours.

6. The method of claim 1, wherein said temperature is between about 1 to 99° C.

7. The method of claim 1, wherein said dispersity index is between about 1.5 and 2.5.

8. The method of claim 1, wherein said treatment is performed under acid conditions at pH between 0 and 7.

9. The method of claim 1, wherein said treatment is performed under basic conditions at pH between about 7 and 14.

10. The method of claim 1, wherein said enzymatic treatment is performed with an enzyme depolymerase.

11. The method of claim 1, wherein said irradiation treatment is performed by x-ray, microwave, UV rays, and gamma irradiation.

12. The method of claim 1, wherein said treatment is a chemical treatment that is performed with an oxidizing or a reducing agent.

13. The method of claim 12, wherein said chemical treatment is carried out with said oxidizing agent under conditions to decrease the molecular weight of said biopolymer.
